(19) United States
(12) Patent Application Publication
(10) Pub. No.: US 2025/0226115 A1
(43) Pub. Date: Jul. 10, 2025

(54) FAIRNESS-AWARE DOMAIN GENERALIZATION FOR MEDICAL DECISION MAKING

(71) Applicant: NEC Laboratories America, Inc., Princeton, NJ (US)

(72) Inventors: Xujiang Zhao, Hillsborough, NJ (US); Haifeng Chen, West Windsor, NJ (US)

(21) Appl. No.: 19/012,201

(22) Filed: Jan. 7, 2025

Related U.S. Application Data
(60) Provisional application No. 63/618,985, filed on Jan. 9, 2024.

(51) Int. Cl. G16H 50/70; G16H 50/20

(52) U.S. Cl. CPC G16H 50/70; G16H 50/20

(57) ABSTRACT

Methods and systems for fairness-aware domain generalization include identifying a sensitive attribute, first features related to the sensitive attribute, and second features irrelevant to the sensitive attribute. Domain-specific information for the first features and the second feature features is decoupled. A classifier is trained with the first features and the second features to ensure cross-domain accuracy while maintaining fairness on the sensitive attribute.

### RELATED APPLICATION INFORMATION

This application claims priority to U.S. Patent Application No. 63/618,985, filed on Jan. 9, 2024, incorporated herein by reference in its entirety.

### BACKGROUND

### Technical Field

The present invention relates to machine learning systems and, more particularly, to generalization of machine learning models in dynamic environments.

### Description of the Related Art

Data distributions for a given application may evolve over time, for example as the underlying systems change and as usage patterns change. In one example, a medical decision making model may need to be updated to reflect changes in scientific consensus and information about new treatments. However, while such models may be updated to improve their accuracy in a new domain, fairness in the handling of sensitive information cannot be disregarded.

### SUMMARY

A method for fairness-aware domain generalization includes identifying a sensitive attribute, first features related to the sensitive attribute, and second features irrelevant to the sensitive attribute. Domain-specific information for the first features and the second feature features is decoupled. A classifier is trained with the first features and the second features to ensure cross-domain accuracy while maintaining fairness on the sensitive attribute.

A system for fairness-aware domain generalization includes a hardware processor and a memory that stores a computer program. When executed by the hardware processor, the computer program causes the hardware processor to identify a sensitive attribute, first features related to the sensitive attribute, and second features irrelevant to the sensitive attribute, to decouple domain-specific information for the first features and the second feature features, and to train a classifier with the first features and the second features to ensure cross-domain accuracy while maintaining fairness on the sensitive attribute.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating a machine learning architecture for disentanglement for counterfactual fairness-aware domain generalization, in accordance with an embodiment of the present invention;

FIG. 2 is pseudo-code for optimizing disentanglement for counterfactual fairness-aware domain generalization, in accordance with an embodiment of the present invention;

FIG. 3 is a block/flow diagram of a method for disentanglement for counterfactual fairness-aware domain generalization, in accordance with an embodiment of the present invention;

FIG. 4 is a block diagram of an exemplary healthcare facility that uses a classifier trained with fairness-aware domain generalization to aid in medical decision making, in accordance with an embodiment of the present invention;

FIG. 5 is a block diagram of a computing device that can train a model with fairness-aware domain generalization, in accordance with an embodiment of the present invention;

FIG. 6 is a diagram of an exemplary neural network architecture that can be used to implement part of the classifier, in accordance with an embodiment of the present invention; and

FIG. 7 is a diagram of an exemplary deep neural network architecture that can be used to implement part of the classifier, in accordance with an embodiment of the present invention.

### DETAILED DESCRIPTION OF PREFERRED EMBODIMENTS

Machine learning models may employ a causal structure framework which implements disentanglement for counterfactual fairness-aware domain generalization. Such models handle data distributions that evolve within dynamic environments and that are influenced by sensitive information. These models provide superior predictive accuracy as compared to models that use exogenous variable disentanglement, and do so while ensuring fairness in the handling of sensitive information. Information may be regarded as sensitive for any appropriate reason, with exemplary types of sensitive information including patient personal information, medical examination information, race, and gender.

To this end, exogenous variables may be partitioned into four categories: semantic information that is caused by sensitive attributes, semantic information that is not caused by sensitive attributes, environmental information that is caused by sensitive attributes, and environmental information that is not caused by sensitive attributes. Among these, the distribution of semantic information remains invariant across domains, whereas the distribution of environmental information varies with changes in the environment. This partitioning makes it possible to disentangle environmental information and sensitive attributes from the embedded representation of classification features, ensuring a reduction in the impact of environmental changes on the model while concurrently upholding its decision fairness.

In this context, the fairness refers to the concept of counterfactual fairness in the field of machine learning. Counterfactual fairness means that predictions or outcomes of the model remain the same if sensitive attributes are altered, holding other variables constant. This approach ensures that the model's predictions are not biased or influenced by sensitive attributes that should not play a role in decision-making. In practice, fairness can be achieved by disentangling causal relationships in the data, ensuring that sensitive information does not affect the predictive outputs in an unfair or unintended manner. For example, in the context of a medical diagnosis, fairness may ensure that the model's predictions are based on medical data rather than being based on patient demographics.

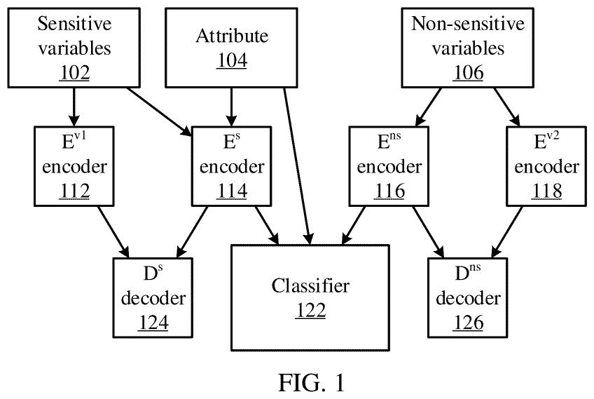

Referring now to FIG. 1, a machine learning architecture for a model 100 is shown. Sensitive variables 102 and non-sensitive variables 106 are considered separately, with separate encoders that focus on environmental and semantic information. A sensitive attribute 104 is used to ensure fairness.

Structural causal models model the causal relationships between variables. A structural causal model may include a directed acyclic graph and a set of structural equations that define the causal relationship among the variables in the graph. The structural equation for an endogenous variable Vᵢ may be expressed as:

Vᵢ=f_{V}_{ᵢ}(Pa_{V}_{ᵢ},U_{V}_{ᵢ})

where Pa_{V}_{ᵢ} denotes the parent set of Vᵢ in the graph and where U_{V}_{ᵢ} denotes a set of exogenous variables that directly affect Vᵢ. The function fᵢ(⋅) represents the causal relationship between the parent variables and Vᵢ. Structural causal models can be used to estimate causal effects and test causal hypotheses. By including sensitive variables 102 in the graph and modeling their causal relationships with other variables, a structural causal model can adjust for sensitivity and can product unbiased estimates of causal effects.

Interventions on structural causal models may change the value of a variable to a specified value. This can be represented mathematically using the do operator, do(Vᵢ=ν). The do operator separates the effect of an invention from the effect of other variables in the system. For example, to investigate the effect of a drug treatment on a disease outcome, the do operator can be used to set the value of a treatment variable to “treated” and observe the effect on the outcome variable. In some cases, the do operator may be represented in terms of two variables Ŷ_{A←a}(U)=P(Ŷ(U)|do(A=a)) for an exogenous variable set U.

Counterfactual fairness models fairness using causal inference tools. Given a predictive problem with fairness considerations, where A, X, Y, and Ŷ represent the sensitive attributes, remaining attributes, the output of interest, and model estimation respectively. A structural causal model [symbol] := [symbol] U, V, F, P(u) [symbol] is given, where V is the set of endogenous variables, P(ν):=P(V=ν)=Σ_{{u|fV(V,u)=ν}}P(u), and U is the set of exogenous variables. The set of deterministic functions F is defined in Vᵢ=f_{V}_{ₛ}(Pa_{V}_{ᵢ}, Uᵥ_{ₜ}).

The predictor Ŷ is counterfactually fair if

$$P ( {\hat{Y}}_{A ← a} ( U ) = y \vert X = x , A = a ) = P ( {\hat{Y}}_{A ← ¬ a} ( U ) = y \vert X = x , A = a )$$

for all y and any value ¬a attainable by A. By setting A to both a and ¬a separately, Ŷ evolves into two distinct variants: Ŷ_{A←a} and ŶA_{←˜a}. From an intuitive perspective, counterfactual fairness seeks to ensure that the values of sensitive attribute A do not influence the distribution of predicted outcome Ŷ.

Classification tasks are considered where the data distribution evolves gradually [symbol] with time. In a training stage, T sequentially arriving source domains [symbol] ={ [symbol] ₁, D₂, . . . , D_{T}} are used, where each domain Dₜ={(xᵢ^{t}, aᵢ^{t}, yᵢ^{t})}ᵢ₌₁ⁿ^{ₜ} is comprised of nₜ labeled samples for t∈{1,2, . . . , T}. And x, a, and y denote data features, sensitive label, and class label respectively. The trained model will be tested on M future domains [symbol] ={D_{T+1}, D_{T+2}, . . . , D_{T+M}}, Dₜ={(xᵢ^{t}, aᵢ^{t}, yᵢ^{t})}ᵢ₌₁ⁿ^{ₜ}(t∈{T+1, T+2, . . . , T+M}), which are not available during training stage. For simplicity, the index i is omitted whenever xᵢ refers to a single data point. The robustness of the model on these unseen domains is enhanced to achieve higher accuracy. Meanwhile, classification fairness is ensured across these M target domains, resulting in the following expression:

$$P ( {\hat{Y}}_{{A}^{t} ← {a}^{t}}^{t} ( U ) = {y}^{t} \vert {X}^{t} = {x}^{t} , {A}^{t} = {a}^{t} ) = P ( {\hat{Y}}_{{A}^{t} ← ¬ {a}^{t}}^{t} ( U ) = y \vert {X}^{t} = {x}^{t} , {A}^{t} = {a}^{t} ) \\ for t ∈ \{ T + 1 , T + 2 , … , T + M \}$$

To achieve the counterfactual generation of p(y|¬a, u) for intervention on A, the exogenous variable U should not contain any part caused by A. Otherwise, there will be situations where intervention on A occurs, but the information caused by A in U remains unchanged, leading to an erroneous generation of y. To address the problem, an effective approach is to define sensitive variables Xₛ⊏X as a subset of features caused by attribute a, whereas non-sensitive variables Xₙₛ⊏X is the other subset of irrelevant features to the intervention.

As an example, the ‘Sex’ attribute in a dataset may be regarded as the sensitive attribute. The characteristics of this attribute can be described as Xₛ={Occupation, Workclass, . . . }, while the remaining features can be denoted as Xₙₛ. Similarly, the exogenous variables of Xₙₛ and Xₛ can be defined to be Uₙₛ and Uₛ, respectively, with Uₛ and Uₙₛ being disentangled. Ideally, Uₛ contains the portion caused by A, rather than the part correlated with A. Therefore, Uₛ should be disentangled from A. On the other hand, Uₙₛ contains only the part correlated with A and does not require decoupling from A.

However, in the face of a constantly changing environment, decoupling the environmental information from Xₛ and Xₙₛ may be needed. To simulate dynamic environments, two variables, U_{ν1} and U_{ν2} may be adopted to capture the dynamic changes in the distributions of Xₛ and Xₙₛ respectively, as they vary with the environments. For the domain [symbol] t at timestamp t, the variables U_{νi} and U_{ν2} are written as U_{ν1}^{t} and U_{ν2}^{t}, respectively.

A causal graph may be built to identify the casual structures between two consecutive domains. Due to the gradual evolution of the environment, a correlation can be identified between the environmental information of each domain and that of the preceding domain. In a system that evolves over time, each domain may be regarded as a different timestamp.

During the inference stage, four distinct encoders to model q(uₛ|xₛ^{t}, a^{t}) as E^{s} encoder 114, q(uₙₛ|xₙₛ^{t}) as E^{ns} encoder 116, q(u_{ν1}|xₛ^{t}) as E^{ν1} encoder 112, and q(u_{ν2}|xₙₛ^{t}) as E^{ν2} encoder 118, respectively. The prior distributions for uₛ and uₙₛ follow standard normal distributions. The environmental variable sequences {U_{ν1}^{t}}ₜ^{T} and {U_{ν2}^{t}}ₜ^{T} can be regarded as two parallel Markov chains (i.e., p(u_{ν1}^{t})=p(u_{ν1}^{t}|u_{ν1}^{<t}) and p(u_{ν2}^{t})=P(u_{ν2}^{t}|u_{ν2}^{<t})). Hence, all the prior distributions are as follows:

$$p ( {u}_{s} ) = ( 0 , I ) ; p ( {u}_{n s} ) = ( 0 , I ) \\ p ( {u}_{v 1}^{t} ) = p ( {u}_{v 1}^{t} \vert {u}_{v 1}^{< t} ) = ( μ ( {u}_{v 1}^{t} ) , {σ}^{2} ( {u}_{v 1}^{t} ) ) \\ p ( {u}_{v 2}^{t} ) = p ( {u}_{v 2}^{t} \vert {u}_{v 2}^{< t} ) = ( μ ( {u}_{v 2}^{t} ) , {σ}^{2} ( {u}_{v 2}^{t} ) )$$

where the distribution p(u_{ν1}^{t}|u_{νi}^{<t}) and p(u_{ν2}^{t}|u_{ν2}^{<t}) can be encoded using recurrent neural networks such as a long-short term memory (LSTM) network. At the initial state t=0 u_{ν1}⁰ and u_{ν2}⁰ are initialized to 0. In the generation phase, all latent variables are fed into two distinct decoders 124 and 126 and a classifier 122 to reconstruct Xₛ, Xₙₛ, and Y.

The decoders 124 and 126 are used to reconstruct the input features Xₛ and Xₙₛ. They ensure that the disentangled latent representations capture the relevant data structure, and they maintain the separation of sensitive and non-sensitive information in the generation phase.

The Kullback-Leibler (KL) divergence KL(q(u|x)∥p(u|x)) can be represented as:

$$K L ( q ( u \vert x ) ‖ p ( u ) ) - {E}_{q ( u \vert x )} [ log p ( x \vert u ) ] + log p ( x )$$

Based on this, an evidence lower bound for a variational autoencoder can be determined as:

$$ELBO = log p ( x ) - K L ( q ( u \vert x ) ‖ p ( u \vert x ) )$$

This means that optimizing the evidence lower bound for a variational autoencoder is equivalent to optimizing KL(q(u|x)∥p(u|x)). Samples from the training domain are denoted as Xₛ^{t} and Xₙₛ^{t} for t∈{1,2, . . . , T}, while the features of samples from the unseen testing domain are represented as Xₛ^{T+m} and Xₙₛ^{T+m} for m≥1.

The KL divergence between q(uₛ, uₙₛ|xₛ^{T+m}, a^{T+m}, xₙₛ^{T+m}) and the unknown domain-invariant ground truth distribution p(uₛ, uₙₛ|xₛ^{T+m}, a^{T+m}, xₙₛ^{T+m}) can be bounded as follows:

$$K L ( q ( {u}_{s} , {u}_{n s} \vert {x}^{T + m} , {a}^{T + m} ) \vert \vert p ( {u}_{s} , {u}_{n s} \vert {x}^{T + m} , {a}^{T + m} ) \leq {inf}_{I ∈ 𝒥} [ {\sum }_{i ∈ I} {β}_{i} ( KL ( q ( {u}_{s} \vert {x}_{s}^{1 : T , i} , {a}^{1 : T , i} ) \vert \vert p ( {u}_{s} \vert {x}_{s}^{t , i} ) ) + KL ( {u}_{n s} \vert {x}_{n s}^{1 : T , i} ) \vert \vert p ( {u}_{n s} \vert {x}_{n s}^{1 : T , i} ) ) ) ] ,$$

$$∀ t ∈ \{ 1 , 2 , … , T \}$$

where xₛ^{1:T,i}, a^{1:T,i} and xₙₛ^{1:T,i} denotes features with index in source domains, J is a feasible set, and βᵢ is a constant. This inequality expresses that the evidence lower bound on the target domains can be optimized by separately optimizing the evidence lower bound concerning Xₛ and Xₙₛ on the source domains.

For any given time point t and domain [symbol] ₜ={(xᵢ^{t}, aᵢ^{t}, yᵢ^{t})}ᵢ₌₁ⁿ^{ₜ}, Uₛ and Uₙₛ may be used to capture the invariant semantic information within the distribution, while U_{ν1}^{t} and U_{ν2}^{t} are utilized to encapsulate the domain-relevant information. Analogous to the variational autoencoder, in this context, q denotes the inference process.

The sensitive attribute A can be used to encode representations containing sensitive information to contribute to the encoding process. Therefore, the evidence lower bound of a sensitive part can be represented as follows:

$${ELBO}_{s} = {\sum }_{t = 1}^{T} \{ {E}_{q ( {u}_{x} \vert {x}_{x}^{t} , {a}^{t} ) q ( {u}_{v 1}^{t} \vert {u}_{+ □1}^{< t} , {x}_{x}^{t} )} [ log p ( {x}_{s}^{t} \vert {u}_{s} , {u}_{v 1}^{t} , {a}^{t} ) ] - KL ( q ( {u}_{s} \vert {x}_{s}^{t} , {a}^{t} ) \vert \vert p ( {u}_{s} ) ) - K L ( q ( {u}_{v 1}^{t} \vert {u}_{v 1}^{t} , {x}_{s}^{t} ) \vert \vert p ( {u}_{v 1}^{t} \vert {u}_{v 1}^{⩽} ) ) \}$$

Like the sensitive part, the evidence lower bound of the non-sensitive part can be represented as follows:

$${ELBO}_{n s} = {\sum }_{t = 1}^{T} \{ {E}_{q ( {u}_{n s} \vert {x}_{n \times }^{t} ) q ( {u}_{v 2}^{t} \vert {u}_{\approx 2}^{t} , {x}_{n s}^{t} )} [ log p ( {x}_{n s}^{t} \vert {u}_{n s} , {u}_{v 2}^{t} ) ] - KL ( q ( {u}_{n s} \vert {x}_{n s}^{t} ) \vert \vert p ( {u}_{n s} ) ) - K L ( q ( {u}_{v 2}^{t} \vert {u}_{v 2}^{t} , {x}_{n s}^{t} ) \vert \vert p ( {u}_{v 2}^{t} \vert {u}_{v 2}^{<} ) )$$

Semantic representations and sensitive attributes are used for classification, with the following loss:

$${ℒ}_{cla} = {E}_{q ( {u}_{x} \vert {x}_{n}^{t} , {a}^{t} ) q ( {u}_{n s} \vert {x}_{nx}^{t} )} [ log p ( y \vert {u}_{s} , {u}_{n s} , {a}^{t} ) ]$$

Taking into account these components, the final evidence lower bound is expressed as:

$$log p ( {x}_{s}^{1 : T} , {x}_{n s}^{1 : T} , {y}^{1 : T} \vert {a}^{1 : T} ) \geq {ELBO}_{s} + {ELBO}_{n s} + {ℒ}_{cla} = ELBO$$

This evidence lower bound is maximized during the training process to render its negative counterpart (−ELBO) a constituent of the objective function.

Counterfactual fairness seeks to minimize the impact of A on the predicted value Ŷ. Therefore, if the following condition is satisfied:

$$p ( \hat{y} \vert a , {u}_{s} , {u}_{n s} ) = p ( \hat{y} \vert ¬ a , {u}_{s} , {u}_{n s} )$$

then the model's predictions attain complete counterfactual fairness. To achieve fairness in classification, it is imperative to augment the objective function with a fairness regularization term:

$${ℒ}_{f} = {\sum }_{t = 1}^{T} {𝔼}_{q ( {u}_{s} \vert {x}_{x}^{t} , {a}^{t} ) q ( y \vert {x}_{nx}^{t} )} [ {‖ p ( y \vert a , {u}_{s} , {u}_{n s} ) - p ( y \vert ¬ a , {u}_{s} , {u}_{n s} ) ‖}_{2} ]$$

Where, for the sake of simplicity, every attribute A is treated as a binary variable in this paper, and ¬a denotes the negation of its original value.

Building upon the analysis of causal structure, Uₛ is concurrently disentangled from both A and Uₙₛ. In other words, Uₛ is simultaneously independent of both A and Uₙₛ (i.e., q(uₛ, a^{t}, uₙₛ)=q(uₛ)q(a^{t}, uₙₛ)). Hence, the disentanglement objective is equivalent to minimizing the KL divergence between q(uₛ, a^{t}, uₙₛ) and q(uₛ)q(a^{t}, uₙₛ)

However, computing this KL divergence directly is infeasible. To address this, a discriminator D is used to output a probability that a set of samples originates from the distribution q(uₛ, a^{t}, uₙₛ) rather than q(uₛ)q(a^{t}, uₙₛ). The KL divergence can then be approximated as follows using the loss function [symbol] about D:

$${ℒ}_{TC} = {\sum }_{t = 1}^{T} K L ( q ( {u}_{s} , {a}^{t} , {u}_{n s} ) \vert \vert q ( {u}_{s} ) q ( {a}^{t} , {u}_{n s} ) ) \approx {\sum }_{t = 1}^{T} {𝔼}_{q ( {u}_{n} , {a}^{t} , {u}_{n s} )} [ log \frac{D ( {u}_{s} , {a}^{t} , {u}_{n s} )}{1 - D ( {u}_{s} , {a}^{t} , {u}_{n s} )} ]$$

To train the discriminator [symbol] _{D} is maximized:

$$= {\sum }_{t = 1}^{T} {𝔼}_{q ( {u}_{x} , {a}^{t} , {u}_{n x} )} [ log ( D ( [ {u}_{s} , {a}^{t} , {u}_{n s} ] ) ) ] + {E}_{q ( {u}_{s} ) q ( {a}^{t} , {u}_{n s} )} [ log ( 1 - D ( [ {u}_{s} , {a}^{t} , {u}_{n s} ] ) ) ] = {\sum }_{t = 1}^{T} {𝔼}_{q ( {u}_{x} , {a}^{t} , {u}_{n x} )} [ log ( D ( [ {u}_{s} , {a}^{t} , {u}_{n s} ] ) ) ] + {E}_{q ( {u}_{s} , {a}^{t} , {u}_{n s} )} [ log ( 1 - D ( perm [ {u}_{s} , {a}^{t} , {u}_{n s} ] ) ) ]$$

where perm[uₛ, a^{t}, uₙₛ] denotes the randomized alteration of the relative sequence between (a^{t}, uₙₛ) and uₛ.

The parameters of all encoders, decoders, and prior networks (LSTMs) are represented as θ, and the parameters of discriminator D are represented as ψ. The training objectives of the model can be summarized into two phases as follows:

$${min}_{θ} {ℒ}_{DCFDG} := - ELBO + {λ}_{f} {ℒ}_{f} + {λ}_{tc} {ℒ}_{TC} , \\ {max}_{ψ}$$

After the completion of training, the trained static feature extractors E^{s} 114 and E^{ns} 116 obtain semantic information (uₛ and uₙₛ). Finally, the classifier 122 is used for prediction by inputting both uₛ and uₙₛ alongside sensitive attribute a.

The classifier in the model predicts the target variable Y, which may be the outcome or label of interest in a supervised learning task. The classifier uses the semantic information (uₛ and uₙₛ) obtained from the static feature extractors, along with the sensitive attribute a, to make these predictions.

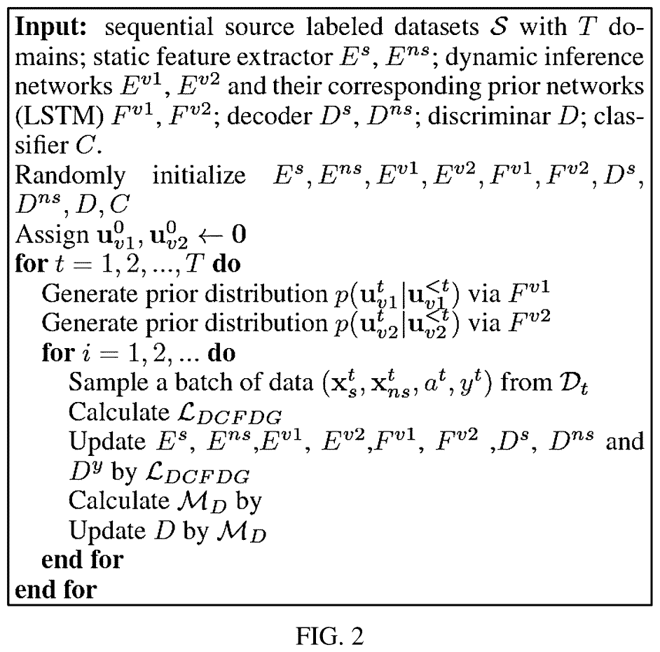

Referring now to FIG. 2, pseudo-code for optimizing with disentanglement for counterfactual fairness-aware domain generalization is shown. Following the description of the model above, this process determines parameters θ and ψ in accordance with the loss functions to generate the framework.

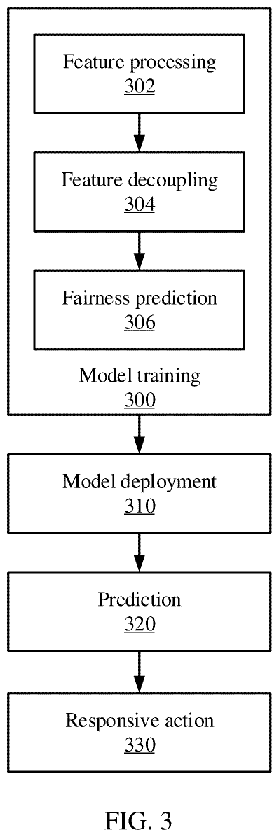

Referring now to FIG. 3, a method for predicting fairness is shown. Block 302 divides the features of each data sample into three parts, particularly including sensitive attributes, caused features, and irrelevant features. Block 304 uses a variational autoencoder to decouple domain-specific information for the caused and irrelevant features. In continuously changing domains, the domain-specific information may be modeled with an LSTM to establish the prior. An adversarial loss is used to ensure the independence between sensitive attributes and the irrelevant features. Block 306 classifies samples for accuracy using the representations of exogenous variables of caused features and irrelevant features, along with the sensitive attribute. A proportion of change when reversing the sensitive attribute is then tested to identify a total effect.

The correlation between features and A can be measured using Pearson product-moment correlation coefficients to assist in the partitioning of block 302. These coefficients may be determined between the sensitive attribute and all three variables. In the case of Xₛ and Xₙₛ, the mean of the coefficients is calculated across all attributes.

In block 304, counterfactual generation of p(y|¬a, u) is used for intervention on A. Block 304 ensures that the exogenous variable U does not contain any part caused by A. Similarly, the exogenous variables of Xₙₛ and Xₛ are defined to be Uₙₛ and Uₛ, respectively, with Uₛ and Uₙₛ being disentangled. Ideally, Uₛ contains the portion caused by A, rather than the part correlated with A. Thus Uₛ needs to be disentangled from A. On the other hand, Uₙₛ contains only the part correlated with A and does not need to be decoupled from A. However, in the face of a constantly changing environment, the environmental information needs to be decoupled from Xₛ and Xₙₛ. To simulate dynamic environments, two variables, U_{ν1} and U_{ν2}, are used to capture the dynamic changes in the distributions of Xₛ and Xₙₛ respectively, as they vary with the environments. For the domain Dₜ at timestamp t, U_{ν1} and U_{ν2} are represented as U_{ν1}^{t} and U_{ν2}^{t}, respectively.

In a training stage 300, the impact of A on the predicted value Y is minimized. During inference, the four encoders and prior distributions for uₛ and uₙₛ follow normal distributions. Where the distribution p(u_{ν1}^{t}|u_{ν1}^{<t}) and p(u_{ν2}^{t}|u_{ν2}^{<t}) can be encoded using recurrent neural networks such as LSTM Wherein, at the initial state when t=0, u_{ν1}⁰ and u_{ν2}⁰ is initialized to 0. In the generation phase, all latent variables are fed into two distinct decoders and a classifier to reconstruct Xₛ, Xₙₛ, and Y. Only environment-independent semantic information is used to reconstruct Y.

Once the model has been trained, the model may be deployed 310 to a target system. In some cases the model may be executed in a same system that trains it, but in some circumstances the model will be transmitted to one or more target systems where live data is available. Block 320 then uses the trained model to perform a prediction, for example classifying newly acquired data. Because the model was trained for fairness, the prediction will be insensitive to private attributes, such as a patient's demographic information. Based on the prediction 320, block 330 performs a responsive action, such as performing a diagnosis of a patient's medical condition and/or determining and administering a treatment.

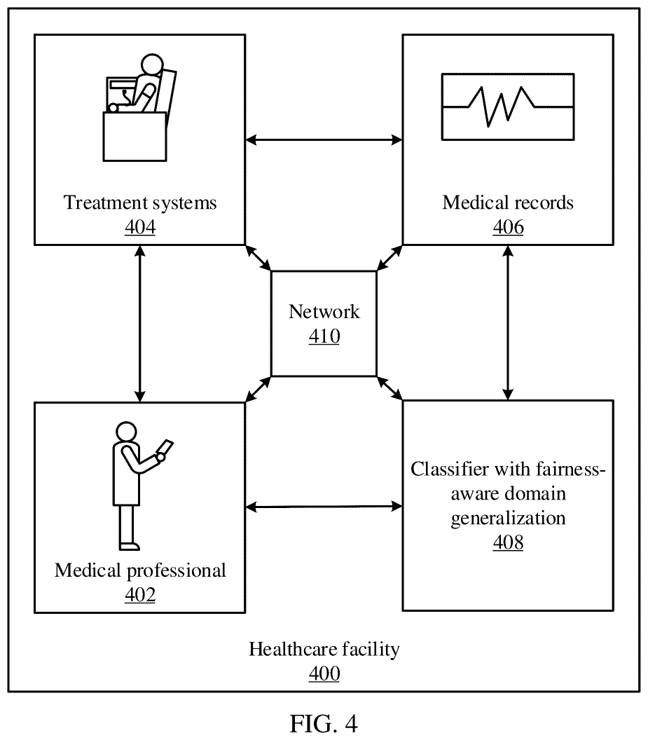

Referring now to FIG. 4, a diagram of information extraction is shown in the context of a healthcare facility 400. A classifier with fairness-aware domain generalization 408 may be used to process information about a patient's medical history to aid with medical decision making and diagnosis. The classifier with fairness-aware domain generalization 408 may be used to generate diagnoses and treatment recommendations relating to a patient's medical condition based on up-to-date medical records 406.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Based on information provided by the classifier with fairness-aware domain generalization 408, the medical professionals 402 may make medical decisions about patient healthcare suited to the patient's needs. For example, the medical professionals 402 may make a diagnosis of the patient's health condition and may prescribe particular medications, surgeries, and/or therapies.

The different elements of the healthcare facility 400 may communicate with one another via a network 410, for example using any appropriate wired or wireless communications protocol and medium. Thus the classifier with fairness-aware domain generalization 408 can receive a query from medical professionals 402 relating to a condition and may formulate a response based on information gleaned from stored medical records 406. The classifier with fairness-aware domain generalization 408 may coordinate with treatment systems 404 in some cases to automatically administer or alter a treatment. For example, if the classifier with fairness-aware domain generalization 408 indicates a particular disease or condition, then the treatment systems 404 may automatically halt the administration of the treatment. Because the classifier with fairness-aware domain generalization 408 has been generalized over changing domains, it can provide accurate diagnoses across a variety of different patient conditions and can handle the evolution of a patient's state over time, while protecting sensitive information.

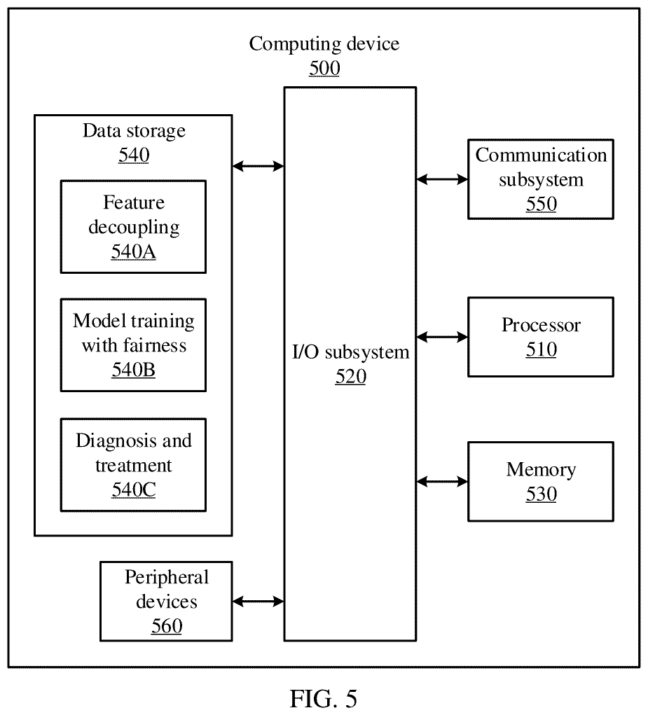

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for determining parameter importance, 540B for pruning a pre-trained model according to parameter importance, and/or 540C for correcting a patient's treatment based on inputs to the model. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

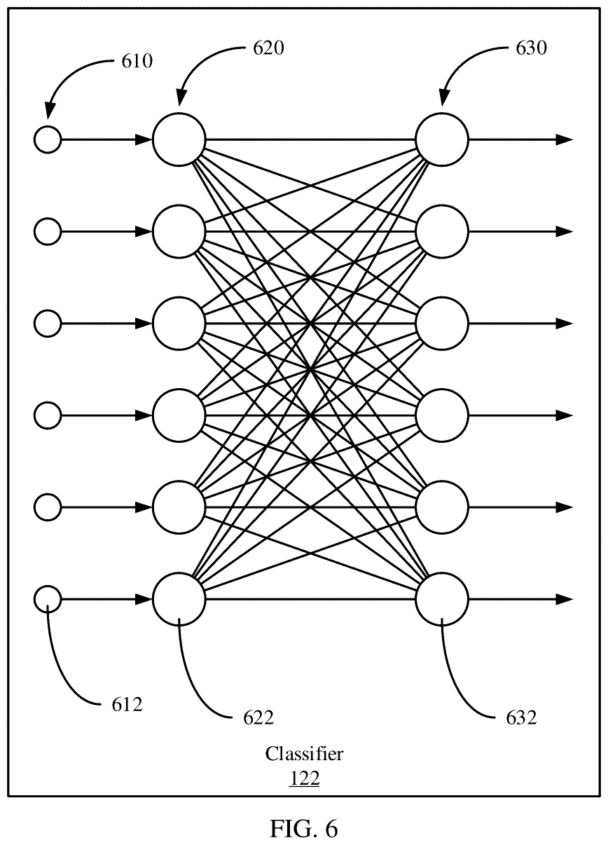

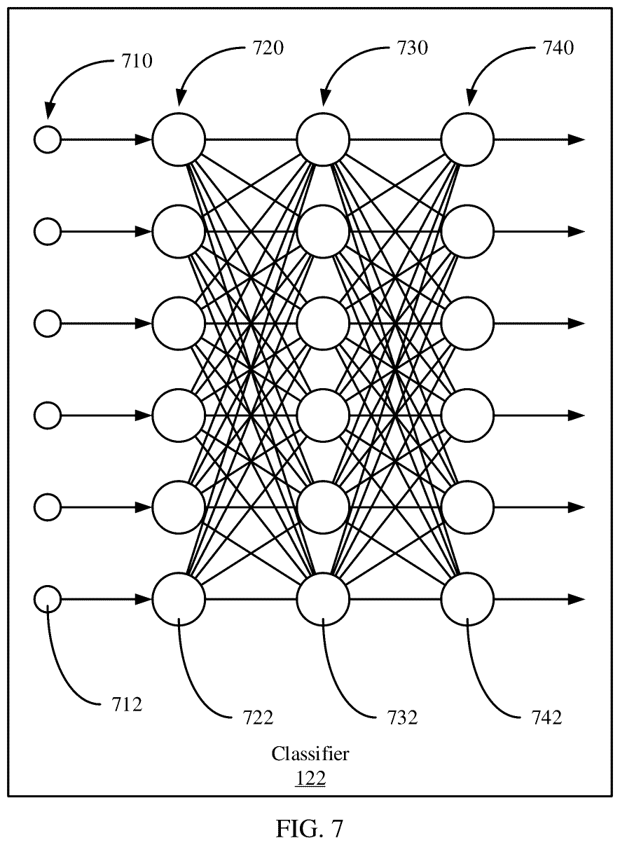

Referring now to FIGS. 6 and 7, exemplary neural network architectures are shown, which may be used to implement parts of the present models, such as the classifier 122. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for fairness-aware domain generalization, comprising:
identifying a sensitive attribute, first features related to the sensitive attribute, and second features irrelevant to the sensitive attribute;
decoupling domain-specific information for the first features and the second feature features; and
training a classifier with the first features and the second features to ensure cross-domain accuracy while maintaining fairness on the sensitive attribute.

2. The method of claim 1, wherein training the classifier includes a variational autoencoder that has a first encoders for the first features, a second encoder for the second features, a third encoder for sensitive exogenous features, and a fourth encoder for non-sensitive exogenous features.

3. The method of claim 1, wherein training includes minimizing an objective function that includes an evidence lower bound based on the first features, an evidence lower bound based on the second features, and a classification loss.

4. The method of claim 3, wherein the objective function further includes a counterfactual fairness loss.

5. The method of claim 4, wherein the counterfactual fairness loss is expressed as a sum over domains of expectation values for predicted values conditioned on the sensitive attribute and a negation of the sensitive attribute.

6. The method of claim 3, wherein the objective function further includes a disentanglement loss.

7. The method of claim 6, wherein the disentanglement loss is approximated as a sum over domains of expectation values based on a discriminator that outputs a probability that a set of samples originates from a distribution defined by the first features, the second features, and the sensitive attribute.

8. The method of claim 1, further comprising using the classifier to diagnose a medical condition of a patient to assist in medical decision making.

9. The method of claim 8, further comprising automatically administering a treatment to the patient based on an output of the classifier.

10. The method of claim 1, wherein the classifier is a machine learning model implemented as a neural network.

11. A system for fairness-aware domain generalization, comprising:
a hardware processor; and
a memory that stores a computer program which, when executed by the hardware processor, causes the hardware processor to:
identify a sensitive attribute, first features related to the sensitive attribute, and second features irrelevant to the sensitive attribute;
decouple domain-specific information for the first features and the second feature features; and
train a classifier with the first features and the second features to ensure cross-domain accuracy while maintaining fairness on the sensitive attribute.

12. The system of claim 11, wherein training the classifier includes a variational autoencoder that has a first encoders for the first features, a second encoder for the second features, a third encoder for sensitive exogenous features, and a fourth encoder for non-sensitive exogenous features.

13. The system of claim 11, wherein training includes minimizing an objective function that includes an evidence lower bound based on the first features, an evidence lower bound based on the second features, and a classification loss.

14. The system of claim 13, wherein the objective function further includes a counterfactual fairness loss.

15. The system of claim 14, wherein the counterfactual fairness loss is expressed as a sum over domains of expectation values for predicted values conditioned on the sensitive attribute and a negation of the sensitive attribute.

16. The system of claim 13, wherein the objective function further includes a disentanglement loss.

17. The system of claim 16, wherein the disentanglement loss is approximated as a sum over domains of expectation values based on a discriminator that outputs a probability that a set of samples originates from a distribution defined by the first features, the second features, and the sensitive attribute.

18. The system of claim 11, further comprising using the classifier to diagnose a medical condition of a patient to assist in medical decision making.

19. The system of claim 18, further comprising automatically administering a treatment to the patient based on an output of the classifier.

20. The system of claim 11, wherein the classifier is a machine learning model implemented as a neural network.
